(19) United States
(12) Patent Application Publication
(10) Pub. No.: US 2004/0180363 A1
(43) Pub. Date: Sep. 16, 2004

(54) Configurable dynamic three dimensional array

(75) Inventors: Lewis Gruber, Chicago, IL (US); David Grier, Chicago, IL (US); Ward Lopes, Chicago, IL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Dymeka Gossett Rooks Pitts PLLC

(21) Appl. No.: 10/735,395

(22) Filed: Dec. 12, 2003

Related U.S. Application Data
(63) Continuation-in-part of application No. PCT/US02/11586, filed on Apr. 12, 2002.

(51) Int. Cl. C12Q 1/68; C12M 1/34

(52) U.S. Cl. 435/6; 536/6; 435/287.2

(57) ABSTRACT

The present invention relates generally to a configurable array of probes for assaying targets within a fluid. The probes are contained within optical traps which allows for alterations in the selection and re-configuration of the quantity or quality of probes in the array. Moreover, the array is dynamic in that once configured the optical traps may allow for independent repositioning of a given optical trap and contained probe.

### BACKGROUND OF THE INVENTION

Throughout this application various publications are referenced within parentheses. The disclosures of these publications in their entireties are hereby incorporated by reference in this application in order to more fully describe the state of the art to which this invention pertains.

1. Field of the Invention

The present invention relates generally to arrays of probes. In particular, the invention relates to a system and method using a plurality of optical traps to form a configurable dynamic array of probes which may or may not be substrate bound.

2. Discussion of the Related Art

Arrays of potentially reactive probes have a long history of use in assays and other chemical and biological tests and experiments. For example, arrays are often used in the fields of genetics, biochemistry, and biology to assay a sample for biological or chemical material (known as a target). Often the sample being assayed is only available in relatively small quantities. This limited availability of some materials led to the development of microarrays useful to present a relatively high density of probes, in a small array, to assay for targets in a small quantity of a sample.

Microarrays used in the testing of biological material are often referred to as bio-chips. Two principal applications of bio-chips are: extraction of sequence information about a specific nucleic acid i.e., whether that nucleic acid corresponds to an organism's entire genome, a singe gene, or a portion of a single gene (U.S. Pat. No. 6,025,136); and evaluation of gene expression. (See Schena, M. et al. “Quantitative monitoring of gene expression patterns with a complimentary DNA microarray,” Science 270 (5235):467-70 (Oct. 20, 1995); D. J. and Winzeler, E. A., “Genomics, gene expression and DNA arrays,” Nature 405(6788):827-836 (2000) and Ekins, R. and Chu, F. W., “Microarrays: their origins and applications;” Trends in Biotechnology 17:217-18 (1999).)

Conventional microarrays are comprised of either a linear or a two-dimensional configuration of oligonucleotide probes, attached to the planar surface of a solid support (substrate). Different types of oligonucleotides are affixed to the substrate at predetermined locations. Consequently, once the microarray is formed, the location of the probes and hence the location of any targets that react with the probes is always known. The attachment of the probe is achieved by either direct synthesis of the oligonucleotide onto the substrate through a process known as in situ photolithography synthesis (U.S. Pat. Nos. 5,837,832 and 5,143,854), or attachment of the oligonucleotide after it has been synthesized.

One drawback of such microarrays is that their linear or two dimensional configuration provides a limited surface area to which probes can be attached, thereby setting a limit on the density of the probes to assay for the targets. In the case of DNA hybridization between targets (DNA or DNA fragments) and probes (immobilized oligonucleotides) the rate of hybridization is controlled by the rate at which the targets are able to pass into contact with the probes. Accordingly, the higher the density of probes, the greater the rate of hybridization.

A second drawback of such microarrays stems from the method of their configuration. Once a microarray is fabricated, the type and quantity of the probes become fixed.

In an alterative approach to assaying for targets in a small quantity of a sample, probes are affixed to the surface of small bead-like substrates. (WO 00/61198 pending for Kambara & Mitsuhashi.) Each bead containing a different probe is marked with a distinct label, thus permitting the identification of each probe and bound target by discerning which bead has what label after completion of the assay (See WO 00/71243).

The identity of the bead and probe is maintained by physically transferring the bead with probe attached into a guide, capillary tube, groove, or holes within a sheet, then washing the beads with targets. While the non-flat nature of the beads does provide greater surface area for the targets to interact then does a microarray probe, the beads must still be held in some pre-determined order throughout the assay to maintain a record of the identity of what bead is supporting which probe or the bead probes must be collected and each bead probe examined after the assay to determine its identity.

An additional drawback of both the microarray and the bead assays is the required physical attachment of the probe to a substitute. In some instances the attachment will in and of itself alter the probe, or affect the process that the probe is being used to assay. In other instances, during or after the initial assay, information may be obtained that would make for desirable alterations of the quality or quantity of the probes, if the identity of the probes was both known throughout the assay and the configuration of the array could be easily altered. However, such alters are not possible with either the microarray or bead assays.

In an unrelated art, it is known to optically trap particles with multiple simultaneously generated optical tweezers. (See generally U.S. Pat. No. 6,055,106 issued to Grier & Durfresne.) Optical tweezers use the gradient forces of a beam of light to trap particles based on the dielectric constant of a particle. To minimize its energy, a particle having a dielectric constant higher than the surrounding medium will move to the region of an optical tweezer when the electric field is the highest.

Other types of traps that can be used to optically trap particles include, but are not limited to, optical vortices, optical bottles, optical rotators and light cages. An optical vortex produces a gradient surrounding an area of zero electric field which is useful to manipulate particles with dielectric constants lower than the surrounding medium or which are reflective, or other types of particles which are repelled by an optical tweezer. To minimize its energy such a particle will move to the region where the electric field is the lowest, namely the zero electric field area at the focal point of an appropriately shaped laser beam. The optical vortex provides an area of zero electric field much like the hole in a doughnut (toroid). The optical gradient is radial with the highest electric field at the circumference of the doughnut. The optical vortex detains a small particle within the hole of the doughnut. The detention is accomplished by slipping the vortex over the small particle along the line of zero electric field.

The optical bottle differs from an optical vortex in that it has a zero electric field only at the focus and a non-zero electric field in all other directions surrounding the focus, at an end of the vortex. An optical bottle may be useful in trapping atoms and nanoclusters which may be too small or too absorptive to trap with an optical vortex or optical tweezer (J. Arlt and M. J. Padgett. “Generation of a beam with a dark focus surrounded by regions of higher intensity: The optical bottle beam,” Opt. Lett. 25, 191-193, 2000.)

The optical rotator is a recently described optical tool which provides a pattern of spiral arms which trap objects. Changing the pattern causes the trapped objects to rotate. (L. Paterson M. P. MacDonald, J. Arlt, W. Sibbett, P. E. Bryant, and K. Dholakia, “Controlled rotation of optically trapped microscopic particles,” Science 292, 912-914, 2001.) This class of tool may be useful for manipulated non-spherical particles and driving MEMs devices or nano-machinery.

The light cage, (Neal in U.S. Pat. No. 5,939,716) is loosely, a macroscopic cousin of the optical vortex. A light cage forms a time-averaged ring of optical tweezers to surround a particle too large or active to be trapped with dielectric constants lower than the surrounding medium. However, unlike a vortex, no-zero electric field area is created. An optical vortex, although similar in use to an optical tweezer, operates on an opposite principle.

There exists a need for an assay method and system in which the interaction of the probes and targets can be evaluated absent attachment of the probe to a substrate. There also exists a need for a method and system of forming an array of probes which is configurable (and re-configurable), the method maintaining the identity of the probes throughout the assay irrespective of the location of the probe. The present invention satisfies these and other needs, and provides further related advantages.

### SUMMARY OF THE INVENTION

The present invention provides a novel and improved method and system to construct, configure and use three dimensional array of probes.

Within a vessel optical traps are generated. The optical traps are produced by directing a beam of light such as a laser beam, at an optical element which alters the beam by patterning its phase to generate beamlets. The beamlets in turn are focused through a lens and produce the gradient conditions necessary for optical trapping. Probes, each with a known characteristic, are then added to the vessel. The probes for a given assay are chosen and than each is selected by containing it within an optical trap.

The quantity and quality of probes forming the array are readily re-configurable by using the optical traps to add, discard, or replace probes. The arrangement of the probes, in the array, relative to one another is also dynamic because the spatial relationship of the probes to one another can be altered while maintaining the identity of the selected probes from which the array was configured. Accordingly, both the array and each of its probes are also movable in three dimensions and can be positioned, moved and re-positioned as a whole, or separately within the vessel.

While a probe remains contained within an optical trap, regardless of whether it has been repositioned with the vessel and regardless of any change in it spatial position “order” in the array, the identity of the probe can be maintained by virtue of knowing the identity of the optical trap by which the probe is contained. Additionally, one optical trap can pass the probe to another optical trap and so on, while tracking the chain of optical trap custody of the probe thereby maintaining the identity of what probe is contained by which optical trap.

Other features and advantages of the present invention will be set forth, in part, in the descriptions which follow and the accompanying drawings, wherein the preferred embodiments of the present invention are described and shown, and, in part will become apparent to those skilled in the art upon examination of the following detailed description taken in conjugation with the accompanying drawings, or may be learned by practice of the present invention. The advantages of the present invention may be realized and attained by means of the instrumentalities and combinations particularly pointed out in the appendant claims.

### DESCRIPTION OF THE DRAWINGS

FIG. 1 illustrates a partial cut-away side view of a system forming an array of configurable probes.

FIG. 2 illustrates a view of a free-probe contained within an optical trap.

FIG. 3 illustrates an overview of a system for forming an array of probes.

FIG. 4 illustrates a beam altering element with multiple static regions.

FIG. 5A illustrates a first operative movement of probes.

FIG. 5B illustrates a second operative movement of probes.

FIG. 6A illustrates a component view of a compact system to form optical traps.

FIG. 6B illustrates an inverted microscope to which the compact system of FIG. 6A attaches.

### DETAILED DESCRIPTION OF THE PREFERRED EMBODIMENTS

Particular embodiments of the invention are described below in considerable detail for the purpose of illustrating its principles and operation. However, various modifications may be made, and the scope of the invention is not limited to the exemplary embodiments described below. For example, while specific reference is made to biological systems and assays for gene sequencing and DNA hybridization, it can be appreciated that the method and system is of equal utility in such areas as optical circuit manufacturing and testing, nanocomposite material construction and testing, fabrication of opto-electronics, electronic components testing, assembly and testing of holographic data storage matrices, chemical assays, genomic assays, proteomics assays, facilitation of combinatorial chemistry, promotion of colloidal self-assembly, and probing non-biological materials.

Certain terminology will be used in the following specification, for convenience and reference and not as a limitation. Brief definitions are provided below:

A. “Beamlet” refers to a sub-beam of light or other source of energy that is generated by directing a beam of light or other source of energy, such as that produced by a laser or collimated output from a light emitting diode, through a medium which diffracts it into two or more sub-beams. An example of a beamlet would be a higher order laser beam diffracted off of a grating.

B. “Phase profile” refers to the phase of light or other source of energy in a cross-section of a beam or a beamlet.

C. “Phase pattering” refers to a patterned phase shift imparted to a beam of light, or a beamlet which alters its phase profile, including, but not limited to, diffracting, phase modulation, mode forming, splitting, converging, diverging, shaping and otherwise steering a section of a beam or a beamlet.

D. “Probe” refers to a biological or other chemical material that selectively binds to, or reacts with, a target. Probes include, but are not limited to, oligonucleotides, polynucleotides, chemical compounds, proteins, peptides, lipids, polysaccharides, ligands, cells, antibodies, antigens, cellular organelles, lipids, blastomeres, aggregations of cells, microorganisms, cDNA, RNA and the like.

E. “Target” refers to a biological or other chemical material whose presence or absence in a sample is detected by binding the target to or reacting the target with a probe. For example, the presence of a target formed of genetic material is detected by a reaction, such as a hybridization reaction, of the genetic material of the target with genetic material of a probe, which possesses the particular characteristic i.e., the complimentary structure, necessary for hybridization. Target materials also include, but are not limited to, oligonucleotides, polynucleotides, chemical compounds, proteins, lipids, polysaccharides, ligands, cells, antibodies, antigens, cellular organelles, lipids, blastomeres, aggregations of cells, microorganisms, peptides, cDNA, RNA and the like.

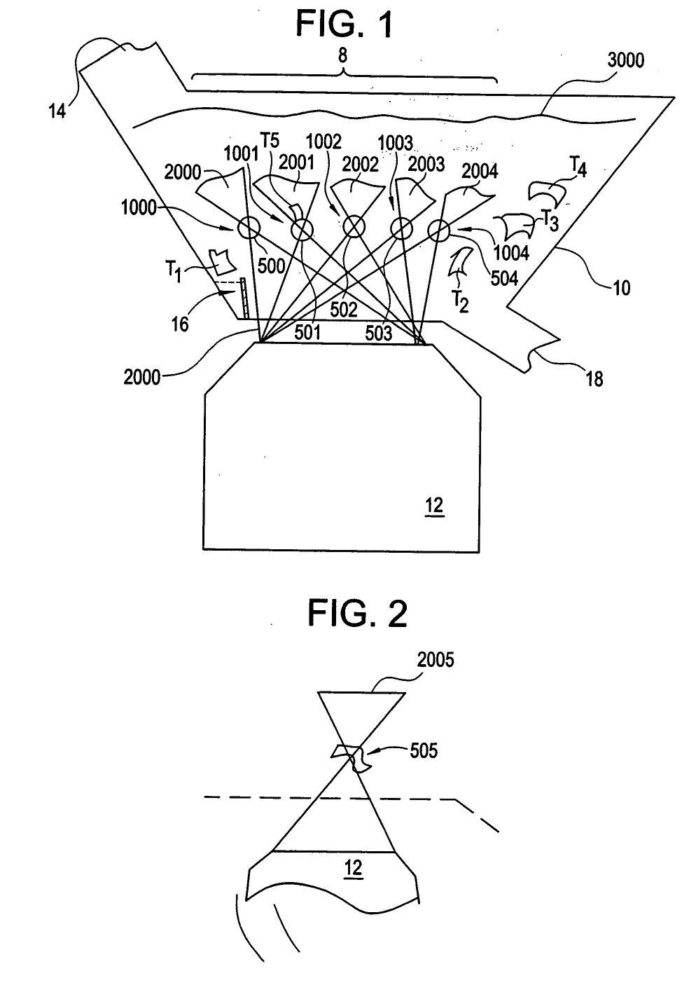

As shown in FIG. 1, the probes 500-504 may be bound to or rated with, any suitable substrate, through any suitable binding process or protocol. An important characteristic of a suitable substrate is that it be a material, which can be contained by, and manipulated with, an optical trap. Representative dielectric substrates include beads, irregular small particles, or other regular small particles. Suitable substrates are constructed of materials, which include, but are not limited to, control pore glass, ceramics, silica, titanium dioxide, latex plastics, such as polystyrene, methylstyrene, polymethyl methacrylate, paramagnetic materials, thoriosol, graphite, Teflon, cross-linked dextrans, such as sepharose, cellulose, nylon, cross-linked micelles, liposomes, and vesicles.

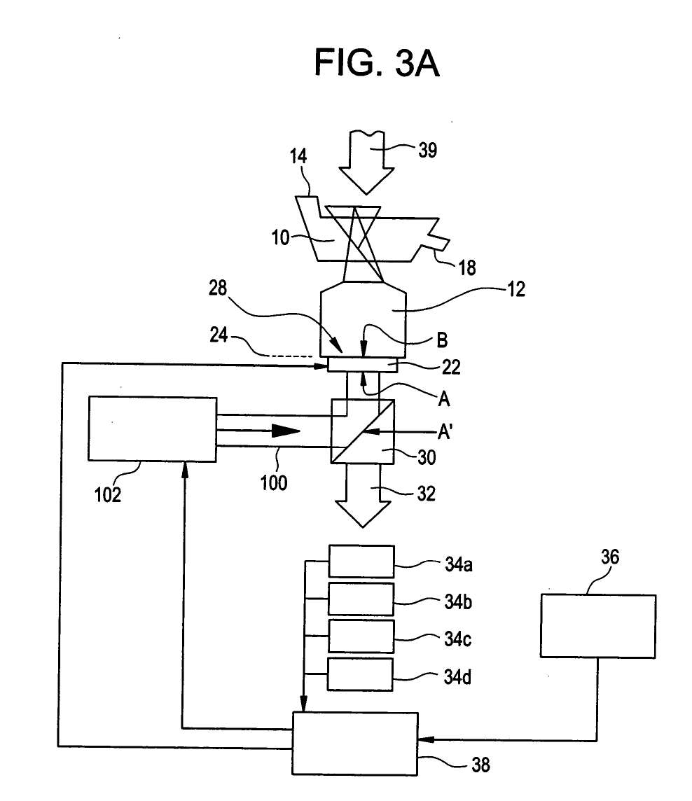

As shown in the alternative embodiment illustrated in FIG. 2, the method of the instant invention also includes using one or using more optical traps 1005 (one shown) to contain one or more probes 505 (one shown) that are unbound to a substrate. It should be understood that the configurable arrays may contain only bound probes, only unbound probes, or a combination of bound and unbound probes. Selection of what mixture, if any, of bound and unbound probes may in part be influenced by a probe's physical properties. Specifically, the properties of certain probes, such as skin cells, may be altered absent adhesion to a substrate. Conversely, the action of other probes, such as proteins, may be better served by maintaining the tertiary structure of the probe/protein by eliminating the substrate.

FIG. 1 illustrates a configurable array 8 of substrate-bound probes 500-504 for assaying a biological material. The probes are configured within a subject cell 10 using movable optical traps 1000-1004 constructed from focused beamlets 2000-2004. The subject cell 10 is a vessel constructed of a substantially transparent material, which allows the beamlets to pass through and which does not interfere with the formation of the optical traps.

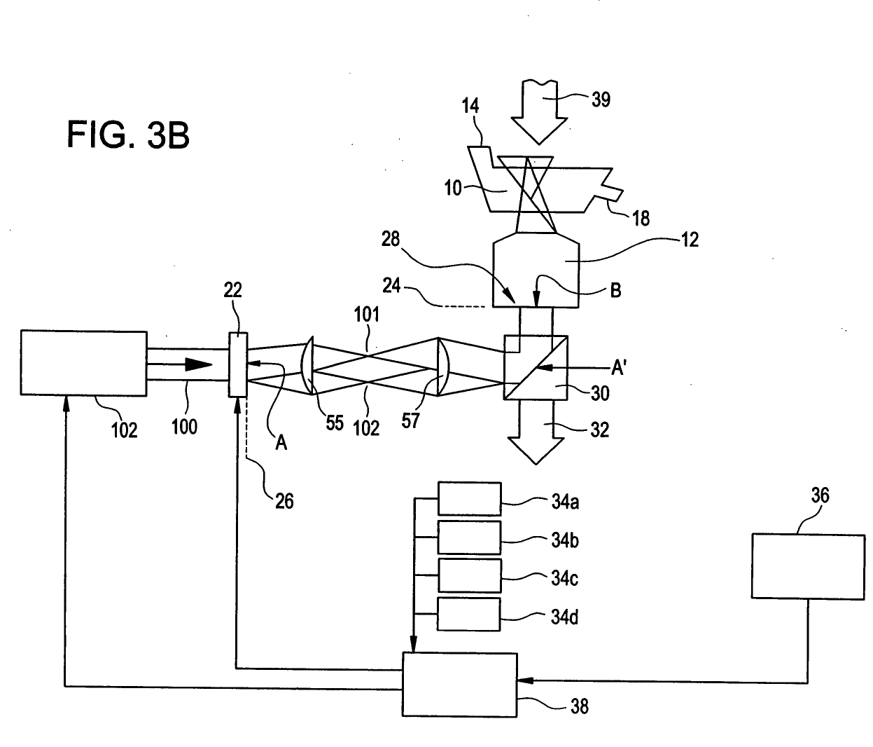

Illustrated in FIG. 3 is an overview of a system to generate and alter the position of the configurable array of probes, generally designated as 20. Movable optical traps 1000-1004 (FIG. 1) are generated within the vessel 10 by passing a collimated light, preferably a laser beam 100, produced by a laser 102 to area A′ on a beam splitter 30. One of the light beams, beam 31, originates from the laser 102 and is redirected so that it proceeds from the area A′ on the beam split 30 to area A on the phase patterning optical element 22. Each beamlet created by the phase patterning optical element 22 then passes through area B at the back aperture 28 of the focusing lens 12. Beamlets are converged by the focusing lens 12 The resulting focused beamlets form the optical traps 1000-1004 by producing the gradient conditions necessary to contain and manipulate the probes in three dimensions. For clarity, only five sets of probes, beamlets, and optical traps are shown in FIG. 1, but it should be understood that a greater or lesser number can be provided depending on the nature, scope, and other parameters of the assay and the capabilities of the system generating the optical traps.

Any suitable laser can be used as the source of the laser beam 100. Useful lasers include solid state laser; diode pumped lasers, gas lasers, dye lasers, alrexanderite lasers, free electron lasers, VCSEL lasers, diode lasers, Ti-Sapphire lasers, doped YAG lasers, doped YLF laser, diode pumped YAG lasers; and flash lamp-pumped YAG lasers. Diode-pumped Nd:YAG lasers operating between 10 mW and 5 W are preferred. The preferred wavelengths of the laser beam 100 used to form arrays for investigating biological material include the infrared, near infrared, visible red, green, and visible blue wavelengths, with wavelengths from about 400 nm to about 1060 nm being most preferred.

The beam splitter 30 is constructed of a dichroic mirror, photonic band gap mirror, omni directional mirror, or other similar device. The beam splitter 30 selectively reflects the wavelength of light used to form the optical traps and transmits other wavelengths. The portion of light reflected from area A′ of the beam splitter is then passed through an area A of an encoded phase patterning optical element 22 disposed substantially in a plane 24 conjugate to a planar back aperture 28 of a focusing lens 12.

When the laser beam 100 directed through the phase patterning optical element 22, the phase patterning optical element produces a plurality of beamlets having an altered phase profile. Depending on the number and type of optical traps desired, the alteration may include diffraction, wavefront shaping, phase shifting, steering, diverging and converging. Based upon the phase profile chosen the phase patterning optical element can be used to generate optical traps in the form of optical tweezers, optical vortices, optical bottles, optical rotators, light cages, and combinations of two or more of these forms.

In those embodiments in which the phase profile of the beamlets is less intense at the periphery and more intense at regions inward from the periphery, overfilling the back aperture 28 by less than about 15 percent is useful to form optical traps with greater intensity at the periphery of the optical traps than optical traps formed without overfilling the back aperture 28.

Suitable phase patterning optical elements are characterized as transmissive or reflective depending on how they direct the focused beam of light or other source of energy. Transmissive diffractive optical elements transmit the beam of light or other source of energy, while reflective diffractive optical elements reflect the beam.

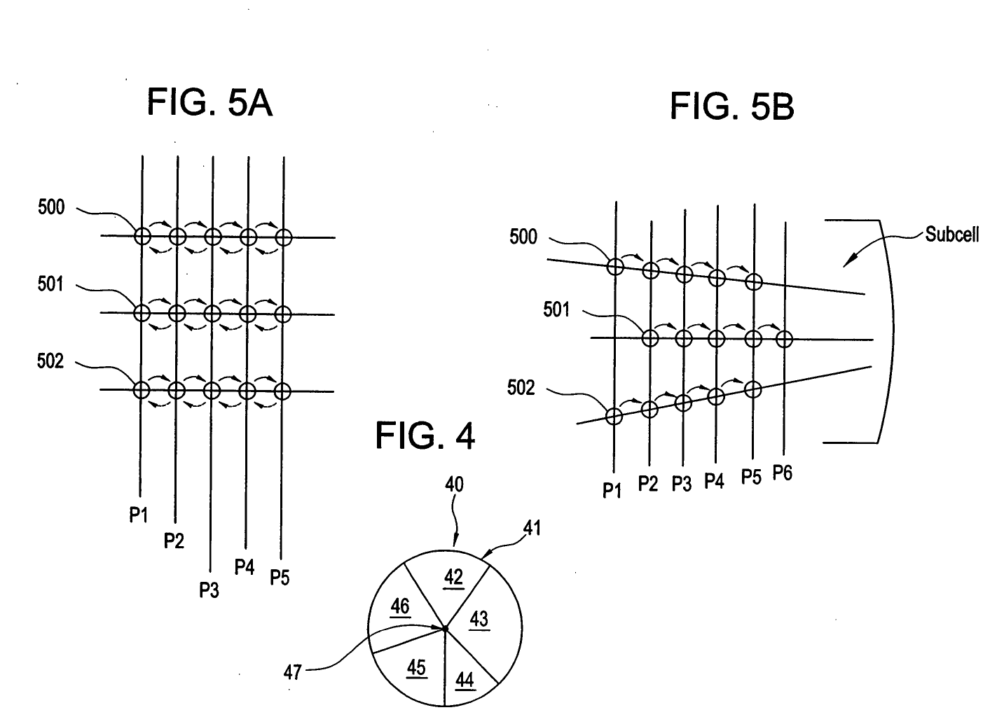

The phase patterning optical element can also be categorized as having a static or a dynamic surface. Examples of suitable static phase patterning optical elements include those with one or more fixed surface regions, such as gratings, including diffractions gratings, reflective gratings, and transmissive gratings, holograms, including polychromatic holograms, stencils, light shaping holographic filters, polychromatic holograms, lenses, mirrors, prisms, waveplates and the like. The static, transmissive phase patterning optical element 40, as shown in FIG. 4, is characterized by a fixed surface 41. However, in some embodiments, the phase patterning optical element itself is movable, thereby allowing for the selection of one more of the fixed surface regions 42-46 by moving the phase patterning optical element relative to the laser beam to select the appropriate region. The static phase patterning optical element may be attached to a spindle 47 and rotated with a controlled electric motor (not shown). The static phase patterning optical element in the embodiment shown in FIG. 4 has a fixed surface 41 and discreet regions 42-46. In other embodiments of static phase patterning optical elements, either transmissive or reflective, the fixed surface 41 has a non-homogeneous surface containing substantially continuously varying regions, or a combination of discreet regions, and substantially continuously varying regions.

Examples of suitable dynamic phase patterning optical elements having a time dependent aspect to their function include computer generated diffractive patterns, phase shifting materials, liquid crystal phase shifting arrays, micro-mirror arrays, including piston mode micro-mirror arrays, spatial light modulators, electro-optic deflectors, accousto-optic modulators, deformable mirrors, reflective MEMS arrays and the like. With a dynamic phase patterning optical element, the medium which comprises the phase patterning optical element encodes a hologram which can be altered, to impart a patterned phase shift to the focused beam of light which results in a corresponding change in the phase profile of the focused beam of light such as diffraction, or convergence. Additionally, the medium can be altered to produce a change in the location of the optical traps. It is an advantage of dynamic phase patterning optical elements, that the medium can be altered to independently move each optical trap.

Preferred dynamic optical elements include phase-only spatial light modulators such as the “PAL-SLM series X7665”, manufactured by Hamamatsu, of Japan or the “SLM 512N15′ and SLM 512SA7,” both manufactured by Boulder Nonlinear Systems of Layafette Colo. The phase patterning optical elements are computer controlled to generate the beamlets 2000-2004 (FIG. 1) by a hologram encoded in the medium which can be varied to the beamlets and select the form of the beamlets.

In some embodiments, the form of the optical traps and/or the locations of optical traps used to form the array are altered and hence configured and re-configured. The form can be changed from its original form to that of an optical tweezer, an optical, a vortex, an optical bottle, an optical rotator or a light cage. The optical trap can be moved in two or three dimensions.

The patterning optical element is also useful to impart a particular topological mode to the laser light, for example, by converting a Gaussian into a Gauss-Laguerre mode. Accordingly, one beamlet may be formed into a Gauss-Laguerre mode while another beamlet may be formed in a Gaussian mode.

The probes are configured within a vessel 10. The vessel 10 is a subject cell constructed of a substantially transparent material, which allows the beamlets to pass through and which does not interfere with the formation of the optical traps. In those embodiments, where the substrate is labeled with a wavelength specific dye, the subject cell should be transparent to the specific wavelength. Furthermore, the subject cell should be constructed of a material that is inert to the substrate. For example, biological substrates such as cells, proteins, and DNA should not stick to the surface of the subject cell and must not be changed or destroyed by the material.

Probes which possess the particular characteristics necessary for binding and/or reacting with the target of interest are selected for addition to the vessel and inclusion in the configurable array. In some of the embodiments, where the probe is bond to a substrate, the substrate is labeled with a marker (such as a wavelength specific dye) to facilitate selection of the probe. In preferred embodiments, all the substrate bound probes that have the same binding or reactivity characteristic are labeled with the same type of markers. When the substrate is labeled with a wavelength specific marker, the selection of probes 500-504 can be accomplished by adding the probes bound to the labeled substrate to the vessel 10. Then, as illustrated in FIG. 3, spectral measurement of the probe's labeled substrate can be used to select (or not to select) a probe for inclusion in the array. In some embodiments (FIG. 2) the probe may be unbound to a substrate and may also be labeled.

In embodiments where unlabeled probes are chosen to form all or part of the array, the probes can be added to the vessel 10 in a sequential order. In sum a case the identity of a probe is known by its load order or a probe's identify can be known on the basis of the time that the probe is added. Alternatively, the probes having binding or reactivity characteristics that differ from one another, can be segregated to different predetermined locations, based on the difference in properties. The probes are then selected on the basis of their location within the vessel.

As seen in FIG. 3, spectroscopy of a sample of biological material can be accomplished with an imaging illumination source 39 suitable for either spectroscopy or polarized light back scattering, the former being useful for assessing chemical identity, and the later being suited for measuring dimensions of internal structures such as the nucleus size. Using such spectroscopic method; in some embodiments, cells are interrogated and the array of probes created from selected interrogated cells. For instance, a computer 38 can be used to analyze the spectral data and to identify suspected cancerous, pre-cancerous and/or non-cancerous cell types. The computer then can apply the information to direct optical traps to contain selected cell types. The contained cells then may be used as the probes in assays, based on the reaction or binding of the contained cells with targets such as other cells, antibodies, antigens, and other biological material, or drugs and other chemicals. Those skilled in the art will recognize that the methodology used to interrogate and concentrate cells based on parameters specific to cancerous cells, may be altered, without departing from the scope of the invention, for use with interrogating and/or separating blastomeres, cells, or other material.

In other embodiments, labeled or unlabeled probes, such as unlabeled probes having differing binding or reactivity characteristics may be placed in a series of sub-cells 16 disposed within the vessel 10. In FIG. 1, for clarity, only one sub-cell is shown. However, it should be understood that a plurality of such sub-cells can be provided. In some embodiments, the boundaries of a sub-cell are constructed with optical traps. A number of optical traps placed in the ring orientation create an optical sub-cell which can perform the same functions as the physical sub-cell 16.

Placement of the probe in a sub-cell 16 is by any suitable means including movement by optical traps, through flow channels, through micro-capillaries or by other equivalent mechanism. In each sub-cell, one or more probes having the same binding or reactivity characteristics are placed. Section of the probes for inclusion in the array is then made on the basis of the sub-cell in which the probe is contained.

The optical traps 1000-1004 are then used to trap the selected probes 500-504 by containing the probes within the optical traps 1000-1004. A group of such contained probes are thereby configured to form an array.

The inventive method and system lends itself to a semi-automated or automated process for tracking the movement and contents of each optical trap. The movement can be monitored, via video camera, spectrum, or an optical data stream and which provides a computer controlling the selection of probes and generation of optical traps information useful to adjusting the type of probes contained by the optical traps and the composition of the probes forming the array. In other embodiments, the movement is tracked based on predetermined movement of each optical trap caused by encoding the phase patterning optical element. Additionally, in some embodiments, a computer is used to maintain a record of each probe contained in each optical trap.

Returning to the beam splitter 30, the beam split 30 also provides a light beam 32 originating from the imaging illumination source 39 which passes through the subject cell 10 forming an optical data stream corresponding to the location of one or more of the beamlets, derived from the location and position of a probe contained by an optical trap.

The optical data stream can then be viewed, converted to a video signal monitored, or analyzed by visual inspection 34a of an operator 36, spectroscopically 34b, and/or video monitoring 34c. The optical data stream 32 may also be processed by a photodetector to monitor intensity, or any suitable device to convert the optical data stream to a digital data stream adapted for use by a computer 38.

To construct the array, the operator 36 and/or the computer 38 will adjust the hologram encoded by the phase patterning optical element 22 to direct the movement of each optical trap to acquire the selected probe and trap it. The plurality of optical traps with contained probes form the composition of the configured array that may be reconfigured as to the composition or position of the probes depending on the needs of the user. Using the optical data stream, the position of one or more of the trapped probes can be identified and their positions monitored. Based on such information, the surface of the phase patterning optical element can be altered, in some embodiments independently, to change the form of one or more of the optical traps containing the probes.

Additionally, the position of one or more of the trapped probes in the array can be tracked by monitoring the position of the optical trap which contains it. Then using such information, any given probe in the array may be independently repositioned within the subject cell by altering the surface of the phase patterning optical element and the identity of each probe remains known by the optical trap in which it is contained irrespective of where the optical trap positions the probe.

In a preferred embodiment, the computer 38 controls the movement of the optical traps both before and after the probes are trapped. In other embodiments, the optical data stream is first converted to a video signal which is then used to produce an image corresponding to the array and the operator views the image to control the movement of at least one of the optical traps based on the image.

Referring to both FIGS. 1 and 3, to perform an assay, a first batch of targets T1-T5 is added to new subject cell 10, which also contains a fluid medium 3000, via an inlet port 14. The array of probes 500-504 is suspended in the medium 3000 via their containment by the optical traps 1000-1004. To increase the opportunity for interaction with the targets T1-T5, the probes may be moved about the subject cell corresponding to movement of the optical traps.

For example, in one embodiment, the probes 500-504 are trolled through the medium 3000 containing the targets T1-T5. By containing the probes optically, as opposed to physically, and moving the probes within the subject cell 10, the opportunity for interaction of a probe with each target is increased, thus improving the speed and efficiency of the assay.

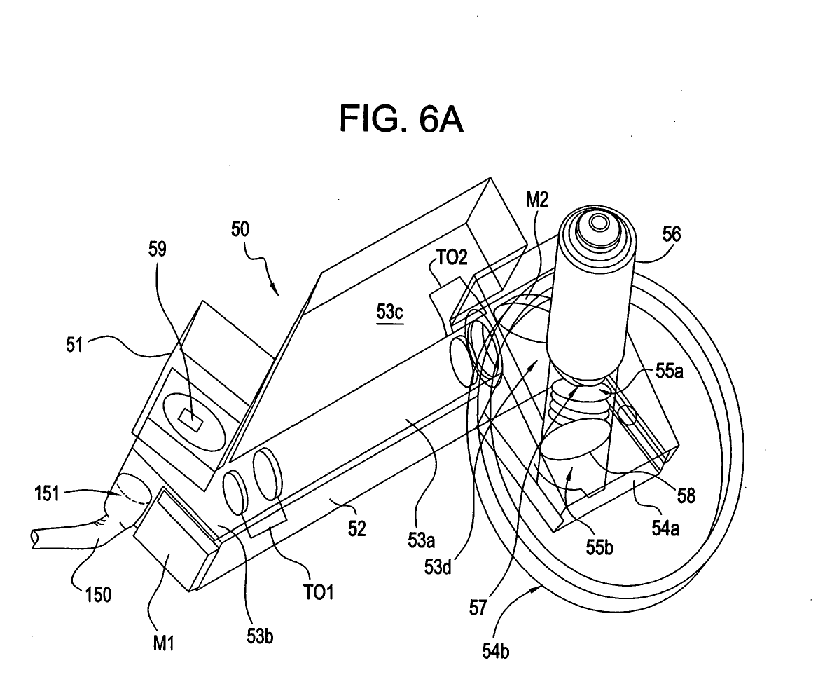

The movement of an array of probes 500-502 via the sequential creation of sets of optical traps is illustrated in FIGS. 5A and 5B. In the embodiment illustrated in FIG. 5A, there is shown a simple linear movement of the array of probes, configured along a line P1 representing a first set of predetermined positions. Movement is accomplished by transferring the probes from a fast set of optical traps to a second, third, and then fourth set. Referring additionally to FIG. 4, the first set of optical traps is generated by directing a laser beam at a first region 42 of the phase patterning optical element 40. When the beamlets emanating from the first region 42 pass through a focusing lens, they form the first set of optical traps at a first position P1 containing the probes 500-503.

To move the probes 500-502 from the first position P1 to a second position P2, the static phase patterning optical element 40 is rotated around a spindle 47 to align the laser beam with a second region 43 which generates the second set of optical traps at a corresponding second set of predetermined positions P2. By constructing the second set of optical traps in the appropriate proximity to the first position P1, the probes can be passed from the first set of optical traps to the second set of optical traps. The sequence may continue passing the probes from the second set of predetermined positions P2 to a third set of predetermined positions P3, from the third set of positions P3 to a fourth set of predetermined positions P4, and from the fourth set of predetermined positions P4 to a fifth set of predetermined positions P5 by the rotation of the phase patterning optical element to align the appropriate region 42-46 corresponding to the desired position P1-P5. The time interval between the termination of one set of optical traps and the generation of the next should be of a duration to ensure that the probes are transferred to the next set of optical traps before they drift away.

Such movement of the probes can be useful to troll the probes through the medium thereby enhancing the opportunity to have targets within the medium interact with the probes. This type of simple movement may also be useful in moving the probes from a sub-cell 16 (FIG. 1) to another area of the subject cell 10, or segregating probes into a sub-cell 16.

In the embodiment illustrated in FIG. 5B there is shown a staggered movement of the probes from a wide to narrow proximity. The staggered movement of the probes occurs in a similar fashion as described in reference to FIG. 5A. However, the first region 42 now produces staggered optical traps with two probes 500 and 502 configured along a line P1, while a third probe 501 is configured at P2, a position between the two probes, but spaced apart from the line P1. As the probes are passed from a first set of optical traps to a second set and moved to second and subsequent positions, the staggered arrangement of the probes allows the probes to be packed densely without placing a set of traps in too close a proximity to two probes at the same time which could cause the probes to be contained by the wrong optical trap.

Once a target has interacted with a probe, spectral methods can be used to investigate the target. The spectrum of those probes which had positive results (i.e., those probes which reacting with or bonded with the targets) can be obtained by using imaging illumination 39 such as that suitable for either inelastic spectroscopy or polarized light back scattering. The computer 38 can analyze the spectral data to identify the desired targets and direct the phase patterning optical element to segregate those desired targets. Those skilled in the art will recognize that the methodology used to segregate targets based on spectral data may be altered, without departing from the scope of the invention, to identify and/or segregate targets based on other information obtained from the targets and/or the optical data stream.

Upon completion of the assay, selection can be made, via computer 38 and/or operator 36, of which probes to discard and which to collect. The reconfigurable nature of the array allows for selective movement of a given optical trap and contained probe. In some cases the medium 3000 and unbound targets will be removed or flushed from the subject cell 10 through an outlet port 18 and the assay will be completed. In other cases, at least some of the probes still contained by optical traps, are reused with additional targets to perform further assays. This technique can be useful in the case of probes that tested positive or negative, depending on the parameters of the assay. In yet other cases, because the array of probes is reconfigurable as to the quantity and characteristics of the probes forming the array, the optical traps can be used to discard unbound probes and acquire additional probes for further experimentation.

In some embodiments, it is not necessary to generate beamlets from each region of the static beam altering optical element 40, or move the beam altering optical element 40 in a set direction. Instead, changing the order of the regions will change the location of the sets of optical traps.

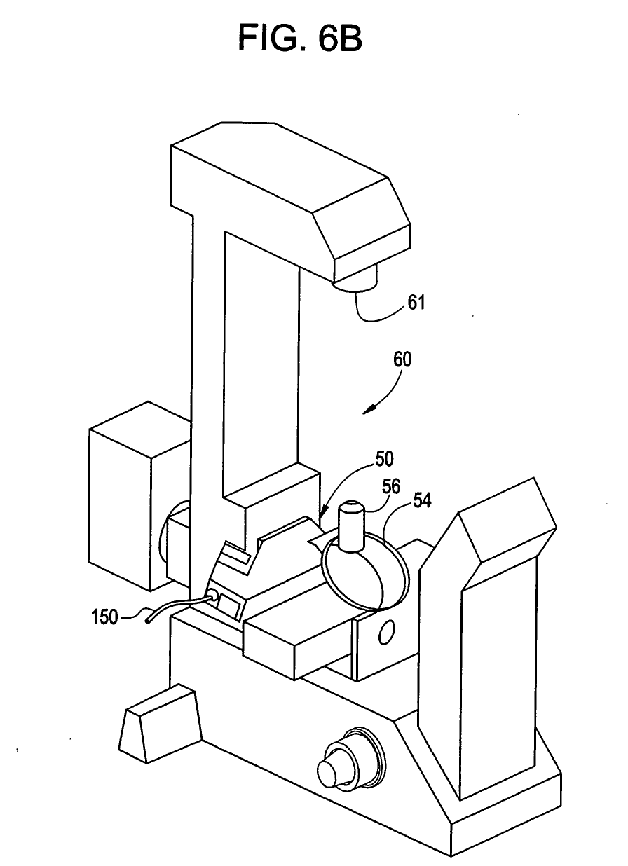

Shown in FIG. 6A is an elevational view of a compact system for forming the optical traps, generally designated 50. The phase patterning optical element 51 is a dynamic optical element, with a reflective, dynamic surface which is also a phase only spatial light modulator such as the “PAL-SLM series X7665,” manufactured by Hamamatsu of Japan the “SLM 512SA7” or the “SLM 512SA15” both manufactured by Boulder Nonlinear Systems of Lafayette, Colo. These dynamic optical elements have an encodable reflective surface in which a computer controls a hologram formed therein.

FIG. 6A shows a compact system for forming the optical traps, the optical element 51 is aligned with, or attached to, a housing 52 through which a first light channel 53a is provided. One end 53b of the first light channel is in close proximity to the optical element 51, the other end 53c of the first light channel intersects with and communicates with a second light channel 53d formed perpendicular thereto. The second light channel is formed within a base 54a of a microscope lens mounting turret or “nosepiece” 54b. The nosepiece 54b is adapted to fit into a Nixon TE 200 series microscope (not shown). The second light channel communicates with a third light channel 55a which is also perpendicular to the second light channel. The third light channel 55a traverses from the top surface of the nosepiece 54b through the base of the nosepiece 54a and is parallel to an objective lens focusing lens 56. The focusing lens has a top and a bottom forming a back aperture 57. Interposed in the third light channel the second light channel and the back aperture 57 of the focusing lens is a dichroic mirror beam splitter 58. Other components within the compact system for forming the optical traps 50 include a first mirror M1, which reflects the beamlets emanating from the phase patterning optical element through the first light channel, a first set of transfer optics TO1 disposed within the first light channel, aligned to receive the beamlets reflected by the first mirror M1, a second set of transfer optic TO2 disposed within the first light channel, aligned to receive the beamlets passing through the first set of transfer lenses TO1, and a second mirror M2, positioned at the intersection of the first light channel and the second light channel, aligned to reflect beamlets passing through the second set of transfer optics TO2 and through the third light channel 55a.

To generate the optical traps, a laser been (not shown) is directed through an optical 150 out a collimator end 151 and reflected off the dynamic surface 59 of the optical element 51. The beam of light (not shown) exiting the collimator end 151 of the optical fiber 150 is diffracted by the dynamic surface 59 of the optical element 51 into a plurality of beamlets (not shown). The number type and direction of each beamlet may be controlled and varied by altering the hologram encoded in the dynamic surface medium 59. The beamlets then reflect off the first mirror M1 through the first set of transfer optics TO1 down the first light channel 53a through the second set of transfer optics TO2 to the second mirror M2; and are directed at the dichroic mirror 58 up to the back aperture 57 of the objective lens 56, are converged through the objective lens 56, thereby producing the optical gradient conditions necessary to form the optical traps. That portion of the light which is split through the dichroic mirror 58, for imaging, passes through the lower portion of the third light channel 55b forming an optical data stream (not shown).

In those embodiments in which the phase profile of the beamlets is less intense at the periphery and more intense at regions inward from the periphery, overfilling the back aperture 57 by less than about 15 percent is useful to form optical traps with greater intensity at the periphery of optical traps than optical traps formed without overfilling the back aperture 57.

Shown in FIG. 6B is an elevational view of a Nixon TE 200 series microscope into which the compact system for forming the optical traps 50 has been mounted, generally designated 60. The nosepiece 54 with the attached a housing 52 fits directly into the microscope via the mount (not shown) for the nosepiece 54a and 54b. The housing and its contents and attached optical element 51 are secured to the nosepiece 54a and 54b require few or no alterations or modifications to the remainder of the microscope. For imaging, an illumination source 61 may be provided above the objective lens 56.

The first and second set of transfer optics TO1 and TO2 are shown containing two lens elements each. The lenses can be either convex or concave. Different and varying types and quantity of lenses such as symmetrical air space singlets, symmetrical air spaced doublets and/or additional lenses or groups of lenses, can be chosen to achieve the image cage transfer from the first mirror M1 to the second mirror M2. In some embodiments the first and second set of transfer optics are symmetrical air spaced doublets, spaced at a distance to act in combination as a telephoto lens.

Since certain changes may be made in the above systems apparatus and methods without departing from the scope of the invention herein involved, it is intended that all matter contained in the above description, as shown in the accompanying drawings specification shall be interpreted in an illustrative, and not a limiting sense.

We claim:

1. A method of configuring and tracking an array of probes comprising;
generating at least two movable optical traps within a vessel;
providing at least two probes within the vessel;
selecting at least two of the probes for inclusion in an array of probes contained within the optical traps;
trapping each of the selected probes with one of the optical traps to configure the array of probes contained within the optical traps; and,
tracking the position of at least one of the trapped probes in the array by monitoring the position of the optical trap which contains it.

2. The method of claim 1, further comprising altering the position of at least one tracked probe by moving the optical trap containing the tracked probe.

3. The method of claim 1, wherein the optical traps are formed of two or more of optical tweezers, optical vortices, optical bottles, optical rotators, or light cages.

4. The method of claim 2, wherein each optical trap is independently movable.

5. The method of claim 2, wherein the movement of each optical trap is controlled by a computer.

6. The method of claim 4, wherein the movement of each optical trap is controlled by a computer.

7. The method of claim 4, wherein at least one of the probes is bound to a substrate labeled with a wavelength specific marker and the at least one bound probe is selected by spectroscopically measuring the marker and using the spectroscopic measurement to select the at least one probe.

8. The method of claim 4, wherein at least two of the probes have binding or reactivity characteristics that differ from one another and at least one of the probes is selected by segregating the probe based on its different binding or reactivity characteristic by moving the probe to a pre-determined location within the vessel and using the location of the segregated probe to select the probe.

9. The method of claim 8, wherein the predetermined location is a physical sub-cell.

10. The method of claim 8, wherein the predetermined location is an optical sub-cell.

11. The method of claim 1 further comprising introducing into the vessel at least one target and determining the reaction or lack thereof of each of the trapped probes with each of the targets.

12. The method of claim 11, wherein the trapped probe is a biological material.

13. The method of claim 11, wherein the trapped probe is a chemical compound.

14. The method of claim 12, wherein the target is a biological material.

15. The method of claim 12, wherein the target is a chemical compound.

16. The method of claim 13, wherein the target is a biological material.

17. The method of claim 13, wherein the target is a chemical compound.

18. The method of claim 12 wherein the trapped probe is an oligonucleotide, a polynucleotide, a protein, a polysaccharide, a ligand, a cell, an antibody, an antigen, a cellular organelle, a lipid, a blastomere, an aggregations of cells, a microorganism, a peptide, cDNA, RNA or combinations thereof.

19. The method of claim 14 wherein the target is an oligonucleotide, a polynucleotide, a protein, a polysaccharide, a ligand, a cell, an antibody, an antigen, a cellular organelle, a lipid, a blastomere, an aggregations of cells, a microorganism, a peptide, cDNA, RNA or a combination thereof.

20. The method of claim 16 wherein the target is selected from one or more of the group consisting of an oligonucleotide; a polynucleotide, a protein, a polysaccharide, a ligand, a cell, an antibody, an antigen, a cellular organelle, a lipid, a blastomere, an aggregations of cells, a microorganism, a peptide, cDNA, RNA or a combination thereof.

21. The method of claim 1 further the probes are all bound to a substrate.

22. The method of claim 1 further comprising the probes are all directly trapped by the optical trap.

23. The method of claim 1 further comprising at least some probes are bound to a substrate and at least some probes are unbound to substrate.

24. The method of claim 21, further comprising altering the position of at least two of the tracked probes in the array by moving the optical traps containing the probes.

25. The method of claim 1, further comprising producing an optical data stream of data corresponding to the identity and position of at least one of the optical traps.

26. The method of claim 24, wherein each optical trap is independently movable.

27. The method of claim 24 wherein the movement of each optical trap is controlled by a computer.

28. The method of claim 25, further comprising receiving the optical data-stream with a computer.

29. The method of claim 28, further comprising analyzing the optical data stream with the computer.

30. The method of claim 29, wherein the computer directs the movement of at least one optical trap based on the analysis of the optical data stream.

31. The method of claim 25, further comprising converting the optical data-stream to a video signal.

32. The method of claim 31, further comprising receiving the video signal with a computer.

33. Th method of claim 32, further comprising analyzing the video signal with the computer.

34. The method of claim 33, further comprising using the computer to direct the movement of one or more optical traps based on the analysis of the video signal.

35. The method of claim 31, wherein the video signal is used to produce an image.

36. The method of claim 35, further comprising an operator viewing the image and directing the movement of one or more optical traps based on the viewing of the image.

37. The method of claim 25, wherein the data is spectroscopic data.

38. The method of claim 37, further comprising using a computer to direct the movement of one or more optical traps based on an analysis of the spectroscopic data.

39. The method of claim 24, wherein the optical traps are formed of two or more of optical tweezers, optical vortices, optical bottles, optical rotators, or light cages.

40. The method of claim 26 wherein the movement of each optical trap is controlled by a computer.

41. The method of claim 24, wherein at least one of the probes is selected by spectroscopically measuring the marker and using the spectroscopic measurement to select the at least one probe.

42. The method of claim 24, wherein at least two of the probes have binding or reactivity characteristics that differ from one another and at least one of the probes is selected by segregating the probe based on its different binding or reactivity characteristic by moving the probe to a pre-determined location within the vessel and using the location of the segregated probe to select the probe.

43. The method of claim 42, wherein the predetermined location is a physical sub-cell.

44. The method of claim 42, wherein the predetermined location is an optical sub-cell.

45. The method of claim 169, wherein the trapped probe is a biological material.

46. The method of claim 169, wherein the trapped probe is a chemical compound.

47. The of claim 46, wherein the target is a biological material.

48. The of claim 46, wherein the target is a chemical compound.

49. The of claim 45, wherein the target is a biological material.

50. The method of claim 45, wherein the target is a chemical compound.

51. The method of claim 45, wherein the trapped probe is an oligonucleotide, a polynucleotide, a protein, a polysaccharide, a ligand, a cell, an antibody, an antigen, a cellular organelle, a lipid, a blastomere, and aggregations of cells, a microorganism, a peptide, cDNA, RNA or combinations thereof.

52. The method of claim 47, wherein the target is an oligonucleotide, a polynucleotide, a protein, a polysaccharide, a ligand, a cell, an antibody, an antigen, a cellular organelle, a lipid, a blastomere, and aggregations of cells, a microorganism, a peptide, cDNA, RNA or a combination thereof.

53. The method of claim 49, wherein the target is selected from one or more of the group consisting of an oligonucleotide, a polynucleotide, a protein, a polysaccharide, a ligand, a cell, an antibody, an antigen, a cellular organelle, a lipid, a blastomere, an aggregations of cells, a microorganism, a peptide, cDNA, RNA or a combination thereof.

54. The method of claim 24 wherein the probes are all bound to a substrate.

55. The method of claim 24 wherein the probes are all unbound to a substrate.

56. The method of claim 24 wherein at least some probes are bound to a substrate and at least some probes are unbound to substrate.

57. A method of assaying biological material comprising:
generating at least two movable optical traps within a vessel;
providing a fluid media in the vessel;
providing at least two probes for biological materials within the fluid media;
selecting at least two of the probes for inclusion in an array;
trapping each of the selected probes with one of the optical traps;
introducing into the vessel at least one target comprised of a biological material; and,
determining the reaction or lack thereof, of each of the trapped probes with each of the targets.

58. The method of claim 57, further comprising tracking the position of at least one of the trapped probes by monitoring the position of the optical trap which contains it.

59. The method of claim 57, wherein the trapped probe is comprised of a biological material.

60. The method of claim 57, wherein the trapped probe is comprised of a chemical compound.

61. The method of claim 59, wherein the trapped probe is an oligonucleotide, a polynucleotide, a protein, a polysaccharide, a ligand, a cell, an antibody, an antigen, a cellular organelle, a lipid, a blastomere, an aggregations of cells, a microorganism, a peptide, cDNA, RNA at combinations thereof.

62. The method of claim 57, wherein the target is an oligonucleotide, a polynucleotide, a protein, a polysaccharide, a ligand, a cell, an antibody, an antigen, a cellular organelle, a lipid, a blastomere, an aggregations of cells, a microorganism, a peptide, cDNA, RNA or a combination thereof.

63. The method of claim 57, further comprising producing an optical data stream of data corresponding to the identity and position of at least one of the optical traps.

64. The method of claim 63 further comprising altering the position of at least one trapped probe in the array by moving the optical trap containing the probe.

65. The method of claim 64, wherein each optical trap is movable independently.

66. The method of claim 64 wherein the movement of each optical trap is controlled by a computer.

67. The method of claim 63 further comprising receiving the optical data-stream with a computer.

68. The method of claim 67 further comprising analyzing the optical data stream with the computer.

69. The method of claim 68 further comprising using the computer to direct the movement of one or more optical traps based on the analysis of the optical data stream.

70. The method of claim 63 further comprising converting the optical data-stream to a video signal.

71. The method of claim 70 further comprising receiving the video signal with a computer.

72. The method of claim 71 further comprising analyzing the video signal with the computer.

73. The method of claim 72 further comprising using the computer to direct the movement of one or more optical traps based on the analysis s of the video signal.

74. The method of claim 70, wherein the video signal is used to produce an image.

75. The method of claim 74 further comprising an operator viewing the image and directing the movement of one or more of the optical traps based on the viewing of the image.

76. The method of claim 63, wherein the data is spectroscopic data.

77. The method of claim 76, further comprising using a computer to direct the movement of one or more optical traps based on an analysis of the spectroscopic data.

78. The method of claim 63 wherein the optical traps are formed of two or more of optical tweezers, optical vortices, optical bottles, optical rotators, and light cages.

79. The method of claim 63 wherein at least one of the probes is bound to a substrate.

80. The method of claim 63 wherein at least one of the probes is unbound to a substrate.

81. The method of claim 79, wherein all the substrate bound probes having the same binding or reactivity characteristic are labeled with the same markers.

82. The method of claim 81, wherein at least one of the markers is a wavelength specific dye.

83. The method of claim 82, wherein at least one of the substrate bound probes is selected by measuring the spectral response of the wavelength specific dye and using the spectral measurement to select the at least one probe.

84. The method of claim 63, wherein at least two of the probes have binding or reactivity characteristics that differ from one another and at least one of the probes is selected by segregating the probe based on its different binding or reactivity characteristic, by moving the probe to a pre-determined location within the vessel and using the location of the segregated probe to select the probe.

85. The method of claim 63, wherein the predetermined location is a physical sub-cell.

86. The method of claim 84, wherein the predetermined location is an optical sub-cell.

87. A mod of configuring an array of probes comprising:
generating at least two movable optical traps within a vessel;
providing at least two probes within the vessel; and,
configuration an array of at least two probes by selecting each probe with one of the optical traps.

88. A method of configuring and reconfiguring an array of probes comprising:
directing a focused beam of light at a phase patterning optical element to form a plurality of beamlets emanating from the phase patterning optical element;
directing the plurality of beamlets at the back aperture of a focusing lens to pass the beamlets through the focusing lens and coverage the beamlets emanating from the focusing lens to generate movable optical traps within a vessel;
providing a plurality of probes within the vessel;
selecting at least two of the probes for inclusion in the array of probes contained within the optical traps;
trapping each of the selected probes with one of the optical traps to configure the array of probes contained within the optic traps; and
altering the position of at least one of the probes contained within the optical traps by moving the optical trap containing the probe to reconfigure the array of probes contained within the optical traps.

89. The method of claim 90 wherein the phase patterning optical element has a static surface.

90. The method of claim 91 wherein the static surface is comprised of two or more discreet regions.

91. The method of claim 90 wherein the position of at least one of the probes contained within the optical traps is altered by changing the discreet region of the static surface to which the beam of light is directed.

92. The method of claim 89 wherein the static surface is substantially continuously

93. The method of claim 89 wherein the position of the at least one optical trap is altered by changing the region of the static surface to which the beam of light is directed.

94. The method of claim 89 wherein the beam altering optical element is a grating a hologram, a stencil, a light shaping holographic filter, a lens, a mirror, a prism, or a waveplate.

95. The method of claim 90 wherein each discreet region is a grating, a hologram, a stencil, a light shaping holographic filter, a lens, a mirror, a prism, or a waveplate.

96. The method of claim 88 wherein the phase patterning optical element is dynamic.

97. The method of claim 96 wherein the position of the at least one of the probes contained in the optical traps is altered by varying the dynamic phase patterning optical element.

98. The method of claim 97 wherein the form of at least one of the optical traps is changed by varying the dynamic phase patterning optical element.

99. The method of claim 97, wherein the changed optical trap is an optical tweezer, a optical vortex, an optical bottle, can optical rotator, or a light cage.

100. The method of claim 91 wherein the form of at least one of the optical traps is changed by moving the discreet static surface.

101. The method of claim 100, wherein the changed optical trap is an optical tweezer, an optical vortex, an optical bottle, an optical rotator, or a light cage.

102. The method of claim 97 wherein the varying of the dynamic phase patterning optical element is a change in a hologram encoded on its surface.

103. A system for forming and tracking optical traps containing probes comprising:
a light source for producing a focused beam of light;
a substantially transparent vessel;
an image illumination source for producing a beam of light illuminating contents of the vessel;
a beam splitter for directing;
a phase patterning optical element for receiving the focused beam of light originating from the lift source and diffracting it into at least two beamlets, the phase patterning optical element having a surface for directing each of the beamlets at a back aperture of a focusing lens, the surface being alterable to change the phase profile and/or orientation of at least one of the beamlets;
the focusing lens for converging each of the beamlets to form optical traps for containing probes; and
a monitor for receiving the beam of light illuminating contents of the vessel and tracking the movement and contents of at least one optical trap.

104. The system of claim 103 further comprising the vessel includes an inlet port.

105. The system of claim 103 further comprising the vessel includes an outlet port.

106. The method of claim 8 wherein the probes are segregated using movement by optical traps flow channels or micro-capillaries.

107. The method of claim 42 wherein the probes are segregated using movement by optical traps, flow channels or micro-capillaries.

108. The method of claim 84 wherein the probes are segregated using movement by optical traps, flow channels or micro-capillaries.

109. The method of claim 63 wherein the targets are selected from one or more of the group consisting of oligonucleotides, polynucleotides, proteins, polysaccharides, ligands, cells, antibodies, antigens, cellular organelles, lipids, blastomeres, aggregations of cells, microorganisms, peptides, cDNA and RNA.

110. The method of claim 9 wherein the probes are segregated using movement by optical traps, flow channels or micro-capillaries.

111. The system of claim 103, wherein the phase patterning optical element is dynamic and which further comprises:
a first computer to control the diffraction by the phase patterning optical element; and,
a second computer to maintain a record of each probe contained in each optical trap.

112. The method of claim 2, wherein the movement of the trapped probes are tracked based on pre-determined movement of each optical trap caused by encoding the phase patterning optical element.

113. A system for forming and tracking optical traps containing probes bound to targets comprising:
a plurality of probes bound to targets;
a light source for producing a focused beam of light;
a substantially transparent vessel:
an image illumination source for producing a beam of light illuminating contents of the vessel;
a beam splitter for directing the beam of focussed light originating from the light source and the beam of light illuminating contents of the vessel;
a phase patterning optical element for receiving the focused beam of light originating from light source and diffracting it into at least two beamlets, the phase patterning optical element having a surface for directing each of the beamlets at a back aperture of a focusing lens, the surface being alterable to change the phase profile and/or orientation of at least one of the beamlets;
the focusing lens for converging each of the beamlets to form optical traps containing the probes bound to the targets; and
a monitor for receiving the beam of light illuminating contents of the vessel and tracking the movement and contents of at least one optical trap.

114. The system of claim 113, wherein the target probe is a biological material.

115. The system of claim 113, wherein the target probe is a chemical compound.

116. The system of claim 114, wherein the target probe is a biological material.

117. The system of claim 114, wherein the target probe is a chemical compound.

118. The system of claim 115, wherein the target probe is a biological material.

119. The system of claim 115, wherein the target probe is a chemical compound.

120. The system of claim 114 wherein the probe is selected from one or more of the group consisting of oligonucleotides, polynucleotides, proteins, peptides, cDNA and RNA.

121. The system of claim 116 wherein the target is selected from one or more of the group consisting of oligonucleotides, polynucleotides, proteins, polysaccharides, ligands, cells, antibodies, antigens, cellular organelles, lipids, blastomeres, aggregations of cells, microorganisms, peptides, cDNA and RNA.

122. The system of claim 118 wherein the target is selected from one or more of the group consisting of oligonucleotides, polynucleotides, proteins, polysaccharides, ligands, cells, antibodies, antigens, cellular organelles, lipids, blastomeres, aggregations of cells, microorganisms, peptides, cDNA and RNA.

123. The method of claim 2 wherein the movement of at least one optical trap is selected from one or more of the group consisting of rotation in a fixed position, rotation in a non-fixed position, movement in two dimension, and movement in three dimensions.

124. The method of claim 2 further comprising moving the optical trap containing the tracked probe by changing the surface of the phase patterning optical element.

125. The system of claim 103 wherein the phase patterning optical element has a static surface.

126. The system of claim 125 wherein the static surface is comprised of two or more discreet regions.

127. The system of claim 126 wherein the static surface is movable to align the focused beam of light with a selected region of the static surface.

128. The method of claim 2 wherein the phase patterning optical element has a static surface having two or more discreet regions and the position of at least one optical trap is altered by changing the discreet region of the static surface to which the beam of light is directed.

129. The system of claim 103 wherein the phase patterning optical element has a substantially continuously varying static surface.

130. The system of claim 127 wherein the phase patterning optical element is selected from the group consisting of gratings, holograms, stencils, light shaping holographic filters, lenses, mirrors, prisms, or waveplates.

131. The system of claim 126 wherein each discreet region is selected from the group consisting of gratings, holograms, stencils, light shaping holographic filters, lenses, mirrors, prisms, or waveplates.

132. The system of 103 wherein the phase patterning optical element is dynamic.

133. The method of claim 2 wherein the phase patterning dynamic element is dynamic and varying the phase patterning optical element alters the position of the at least one optical trap.

134. The method of claim 4 wherein the phase patterning dynamic element is dynamic and varying the phase patterning in optical element alters the position of the at least one optical trap.

135. The method of claim 2, wherein the phase dynamic element is dynamic and varying the phase patterning optical element changes the form of at least one of the optical traps to an optical tweezer, an optical vortex, an optical bottle, an optical rotator, or a light cage.

136. The method of claim 2 wherein the phase patterning optical element has a static surface having two or more discreet regions and the form of at least one of the optical traps is changed by moving the static surface.

137. The method of claim 136, wherein the form of the changed optical trap is selected from the group consisting of optical tweezers, optical vortices, optical bottles, optical rotators, and light cages.

138. The system of claim 132 wherein the phase patterning optical element is selected from at least one of the group consisting of variable computer generated diffractive patterns, phase shifting mater, liquid crystal phase shifting arrays, micro-mirror assays, spatial light modulators, electro-optic deflectors, accousto-optic modulators, deformable mirrors and reflective MEMS arrays.

139. The system of claim 132 further comprising a computer to control the dynamic phase patterning optical element.

140. The system of claim 103, further comprising a sub-cell within the vessel for segregating at least one of the prove-containing optical traps.

141. The system of claim 140 wherein the sub-cell is a physical sub-cell.

142. The system of claim 150, further comprising a computer to alter the phase patterning optical element to change the orientation of at least one of the beamlets and move the corresponding optical trap in order to contain the probe.

143. The system of claim 103 wherein the light source is a laser for producing a focused beam with a wavelength in the green spectrum.

144. The system of claim 103 wherein the light source is a laser for producing a focused beam with a wavelength it the visible blue spectrum.

145. The system of claim 103 wherein the light source is a laser for producing a focused beam with a wavelength in the visible red spectrum.

146. The system of claim 103 wherein the light source produces a focused beam of light having a wavelength in the range of about 400 nm to about 1060 nm.

147. The system of claim 103 wherein the light source is a laser beam.

148. The system of claim 103 further comprising a computer for receiving the optical data-stream.

149. An apparatus to form an army of optical traps comprising:
a light source for producing a focused beam of light;
a focusing lens having a top and bottom, the bottom forming a back aperture;
a phase patterning optical element for receiving the focused beam of light and diffracting it into at least two beamlets, the phase patterning optical element having a surface for directing each of the beamlets at the back aperture of the focussing lens;
a first channel having first and second ends, the first end in communication with the phase patterning optical element;
a second light channel having first and second ends, the first end intersecting the second end of the first light channel;
a third light channel having first and second ends, the first end in communication with the second end of the second light channel;
a first mirror reflecting the beamlets emanating from the phase patterning optical element through the first light channel;
a first set of transfer optics disposed within the first light channel, aligned to receive the beamlets reflected by the first mirror;
a second set of transfer optics disposed within the first light channel, aligned to receive the beamlets passing through the first set of transfer lenses;
a second mirror positioned at the intersection of the first light channel and the second light channel, aligned to reflect beamlets passing through the second set of transfer optics through the third light channel; and
a third mirror disposed within the third light channel for reflecting beamlets passing through the third light channel to the back aperture of the focusing lens and forming an array of optical traps.

150. The apparatus of claim 149 further comprising an illumination source for producing a beam of illuminating light disposed next to the top of the focusing lens.

151. The apparatus of claim 150, wherein the third mirror is a dichroic beam splitter for directing the beam of focussed light originated from the light source and the beam of light originating from the illumination source.

152. The apparatus of claim 149 wherein each set of transfer optics is selected from the group consisting of symmetrical air spaced singlets and symmetrical air speed doublets.

153. The apparatus of claim 149 wherein each set of transfer optics is comprised of lenses selected from the group consisting of convex lens and concave lenses.

154. The apparatus of claim 149 wherein the first and second sets of transfer optics are symmetrical air spaced and are spaced at a distance to act in combination as a telephoto lens.

155. The method of claim 25 further comprising introducing into the vessel at least one target and determining the reaction or lack thereof of each of the trapped probes with each of the targets.

156. A method in accordance with claim 1 further comprising moving at least one of the trapped probes by transferring the probe from one optical trap to another.

157. A method in accordance with claim 1 further comprising moving at least three of the trapped probes by transferring the probe from a first set of optical traps to a second set of optical traps.
